(19) United States
(12) Patent Application Publication
(10) Pub. No.: US 2005/0143392 A1
(43) Pub. Date: Jun. 30, 2005

(54) Chrysogenazine obtained from fungus Penicillium chrysogenum having antibacterial activity

(75) Inventors: Chandrakant Govind Naik, Goa (IN); Prabha Devi, Goa (IN); Ely Rodrigues, Goa (IN)

Correspondence Address: WOMBLE CARLYLE SANDRIDGE & RICE, PLLC, P.O. BOX 7037, ATLANTA, GA, 30357-0037, US

(73) Assignee: Council of Scientific & Industrial Research, New Delhi (IN)

(21) Appl. No.: 10/804,201

(22) Filed: Mar. 19, 2004

(30) Foreign Application Priority Data
Dec. 31, 2003 (WO) PCT/IN03/00435

(51) Int. Cl. C07D 43/02; A61K 31/496

(52) U.S. Cl. 514/254.09; 544/373

(57) ABSTRACT

The present invention relates a novel compound, chrysogenazine containing both indole and diketopiperazine ring systems, isolated from the chloroform fraction of the fermentation broth of Penicillium chrysogenum and the gross structure of the compound was elucidated by a detailed analysis of spectroscopic data (IR, NMR, MS), in addition, this invention also assesses the biological activity of the compound which reveals its antibacterial activity against the human pathogen, Vibrio cholerae, demonstrated by the disc diffusion assay.

### FIELD OF THE INVENTION

The present invention relates to the extraction, isolation and identification of a new compound 3,1′-didehydro-3[2″(3′″,3′″-dimethyl-prop-2-enyl)-3″-indolyl methylene]-6-methylpiperazine-2,5-dione, as shown in FIG. 1; containing an indole and a diketopiperazine moiety from a mangrove-associated fungus, Penicillium chrysogenum, and is designated as chrysogenazine from the specific name of the fungus chrysogenum. This invention also describes the process involved in its isolation and evaluates its antibacterial properties against the human pathogen Vibrio cholerae MCM B-322.

### BACKGROUND OF THE INVENTION

Recent years have seen a growing interest in the discovery of metabolites from associated micro-organisms due to the speculation that a number of metabolites obtained from marine plants and invertebrates may be produced by associated micro-organisms.

Penicillium chrysogenum is a known penicillin producer (Ariyo et al, 1998). The antibacterial effect of penicillin was discovered by Alexander Fleming in 1929, which became a “wonder drug” which saved millions of lives. It is still a “front-line” antibiotic, although the development of penicillin-resistance in several pathogenic bacteria now limits its effectiveness. P. Chrysogenum is also 44known to yield hexaketides sorbicillin (Trifonov et al., 1983) and chrysogine, 2-(α-hydroxyethyl-4(3H)quinazolinone (Bergman and Brynolf, 1990). In lactose containing media it is known to synthesize β-galactosyl oligosaccharides (Ballio and Russi, 1960).

The increasing incidence of drug resistance in pathogenic microbes as well as the increasing frequency of infectious diseases in immunocompromised individuals necessitates the discovery of new anti-infective agents.

The 2,5-DKP (Diketopiperazine), head-to-tail dipeptide dimers, are a common naturally occurring structural motif. They are known to be frequently generated as unwanted by-products or degradation products in the syntheses of oligopeptides (Dinsmore and Beshore, 2002). Some piperazine derivatives are reported to exhibit activities towards the central nervous systems, such as anti-anxiety activity and anti-convulsive activity, as described in U.S. Pat. No. 3,362,956. Piperazine derivatives are also known to possess calmodulin inhibitory activity as reported in Arzneim Forsch., (1987). Some of the compounds with calmodulin inhibitory activity has been revealed to be antihypertensive and vasodilatory in action (U.S. Pat. No. 5,681,954).

In view of the above factors, the present invention describes a novel compound, which is a DKP derivative from an associated fungus Penicillium chrysogenum. The present invention also demonstrates its potentials against human pathogen Vibrio cholerae. Natural penicillin obtained from culture filtrates of Penicillium notatum or Penicillium chrysogenum are penicillin G and penicillin V. Both these are active against Gram-positive bacteria and not against Gram-negative species. However, our invention has isolated an antibiotic from Penicillium chrysogenum, which is active against Vibrio cholerae which is Gram-negative, rod shaped bacteria causing cholera in humans.

Vibrio cholerae is known to produce cholera toxin, whose action on the mucosal epithelium is responsible for the characteristic diarrhea of the disease cholera. Tetracycline is still the first choice for bacterial infection causing cholera. The emergence of bacterial resistance to tetracycline has limited the use of these agents. In addition, tetracyclines are strong chelating agents. This ability to chelate to metals, such as calcium, results in tooth discoloration when it is administered in children. For the above reasons, chrysogenazine will prove to be a commercially potential alternate source for the above disease in humans.

Since vibrios mostly occur in the surface waters (both marine and fresh water habitats) and are associated with aquatic animals, transmission to humans is by water or food. Thus cholera can smoulder in an endemic fashion on the subcontinent. Cholera was reported for the first time in South America (1991), in Peru, the outbreaks quickly grew to epidemic proportions and spread to other South American countries, Central American countries, Mexico etc. Outbreaks were also reported from Bangladesh, India etc. Therefore, commercialisation of this drug will have potential market in all developed and developing countries where cholera epidemic is a serious problem.

### OBJECTS OF THE INVENTION

The principal object of the present invention is to isolate a novel compound from the fermentation broth of Penicillium chrysogenum.

Another object of the present invention is to provide a process for the isolation of the compound.

Yet another object of this invention is to identify the antibacterial activity of the compound against the human pathogen Vibrio cholerae.

### SUMMARY OF THE INVENTION

In summary, the present invention provides a process for obtaining substantially pure and novel chrysogenazine from the fermentation broth of P. chrysogenum as a yellow solid. This novel compound contains an indole and a diketopiperazine moiety and shown in FIG. 1. In addition, this compound mentioned herein show antibacterial properties against the human pathogen Vibrio cholerae.

### BRIEF DESCRIPTION OF THE ACCOMPANYING TABLE

Table 1: NMR data of chrysogenazine (300 MHz, CDCl₃)

### BRIEF DESCRIPTION OF THE ACCOMPANYING FIGURES

FIG. 1: Structure of chrysogenazine

FIG. 2: ¹H NMR spectrum of chrysogenazine

FIG. 3: ¹³C NMR spectrum of chrysogenazine

FIG. 4: IR spectrum of chrysogenazine

FIG. 5: MS data of chrysogenazine

### BRIEF DESCRIPTION OF THE ACCOMPANYING PLATE

Plate 1: Antibacterial activity of chrysogenazine using simple disc diffusion technique (inhibition zone of 4-5 mm diameter).

### DETAILED DESCRIPTION OF THE INVENTION

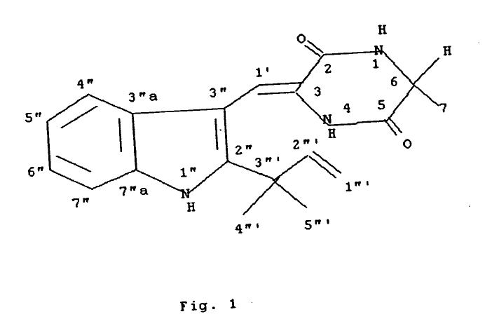

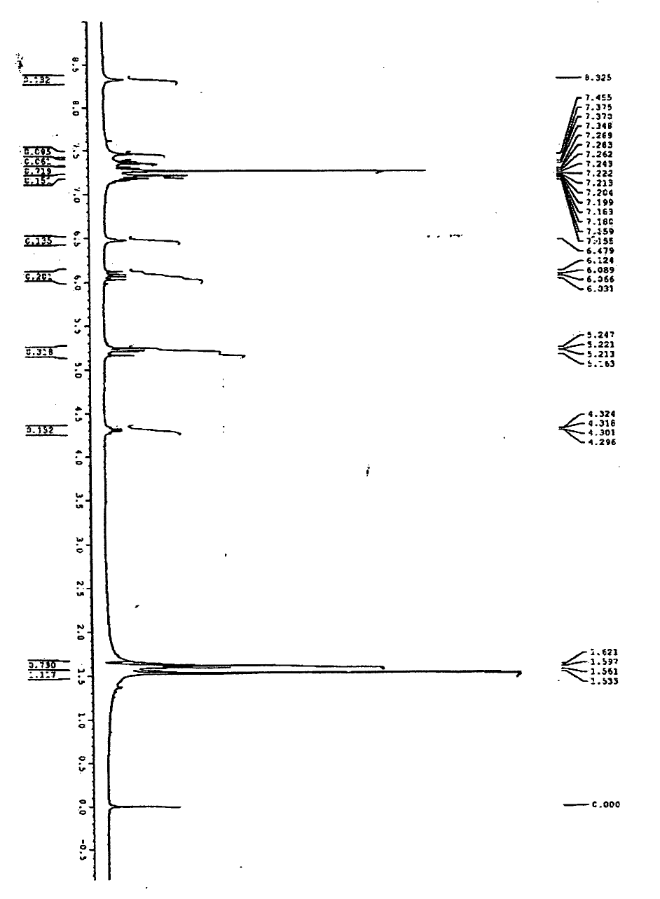

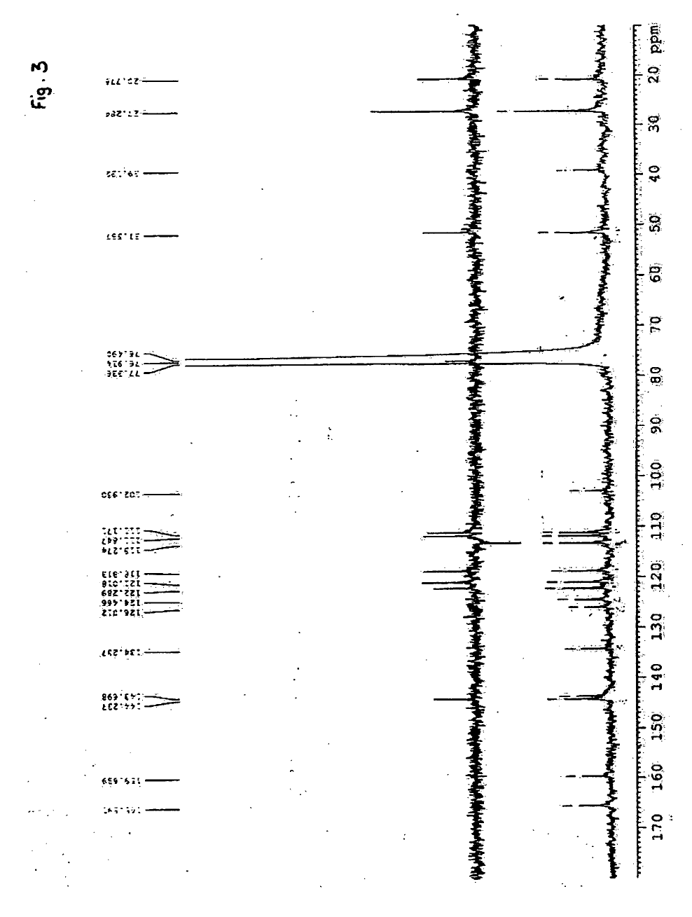

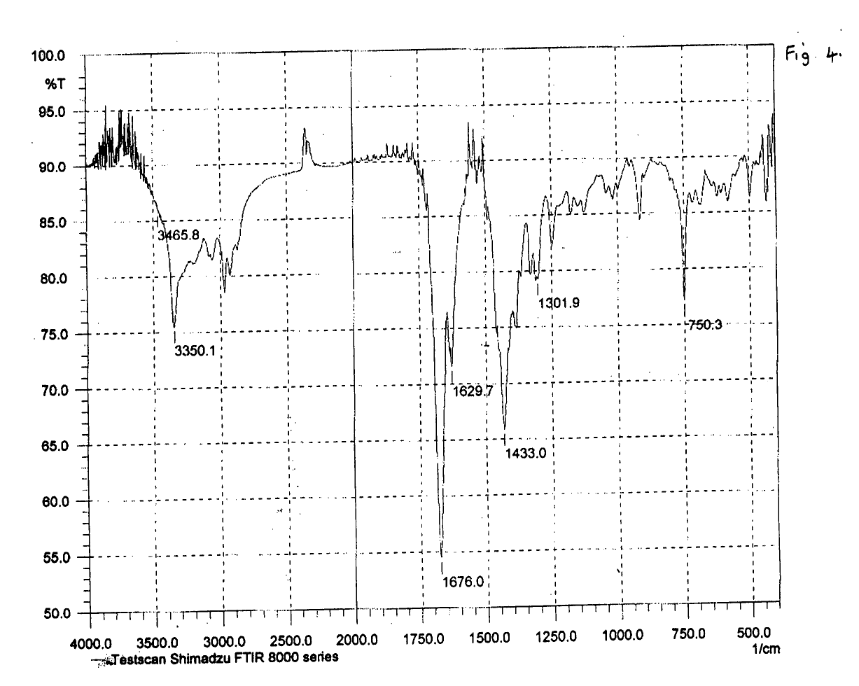

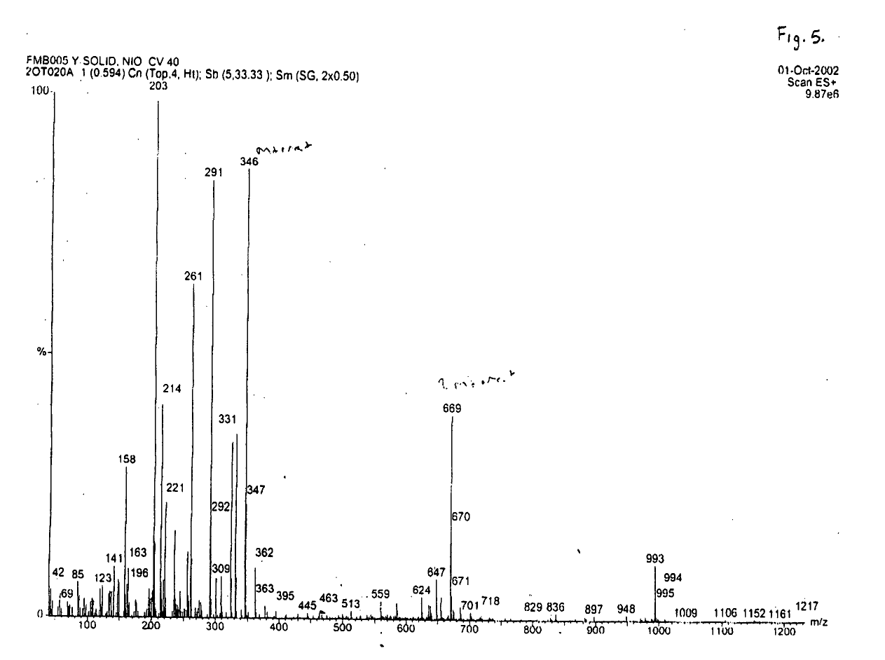

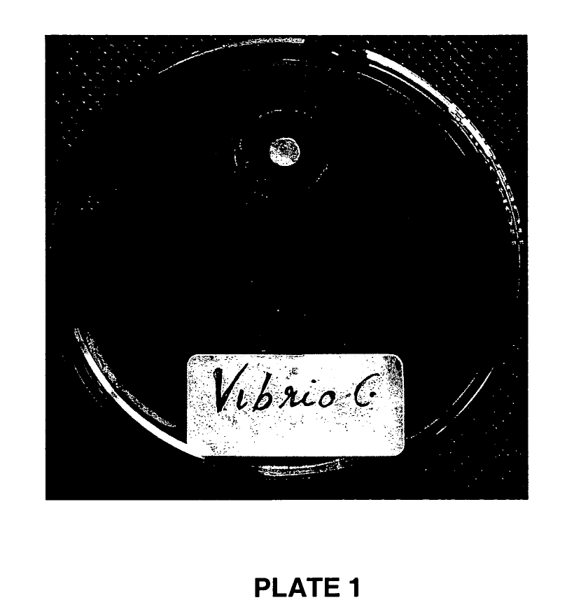

Accordingly, the present invention provides a novel diketopiperazine derivative affective against human pathogen, Vibrio cholerae. The compound 3,1′-didehydro-3[2″(3′″,3′″-dimethyl-prop-2-enyl)-3″-indolyl methylene]-6-methylpiperazine-2,5-dione of the present invention containing both an indole and a diketopiperazine moiety has been designated as chrysogenazine from the specific name of the fungus chrysogenum and given in FIG. 1. This compound has the NMR assignments as given in Table 1, when recorded in CDCl₃ and DMSO.

TABLE 1

| | | | HMQC | |
|---|---|---|---|---|
| Position | δ ¹H | δ ¹H^{a} | ¹H—¹³C | HMBC |
| 1 | 6.43(1H, brs) | 8.32(brs) | — | — |
| 2 | — | — | 159.6s | — |
| 3 | — | — | *102.0 | — |
| 4 | 7.4(1H, brs) | 8.6(brs) | — | — |
| 5 | — | — | 165.5s | — |
| 6 | 4.25(1H, dd, J=6.9Hz | 4.15(dd, J=6.9Hz) | 51.5d | 20.7, 159.6 |
| 7 | 1.55(3H, d, J=7.2Hz) | 1.43(d, J=6.9Hz) | 20.7q | 165.5 |
| 1′ | 7.16(1H, s) | 6.96(s) | 111.8d | 126, 143.6, 159.6 |
| 1″ | 8.2(1H, brs) | 11.06(brs) | — | — |
| 2″ | — | — | 143.6s | — |
| 3″ | — | — | *124.4s | — |
| 3″a | — | — | 126.0s | — |
| 4″ | 7.36(1H, m, J=6.6, 1.5Hz) | 7.47(1H, d, J=7.8Hz) | 111.1d | 122, 134.2 |
| 5″ | 7.11(1H, m) | 7.06(1H, t, J=7.2Hz) | 121.0d | — |
| 6″ | 7.27(1H, m) | 7.14(1H, t, J=7.2Hz) | 122.0d | — |
| 7″ | 7.2(1H, m) | 7.25(1H, d, J=7.5Hz) | 118.8d | — |
| 7″a | — | — | 134.2s | — |
| 1′″ | 5.16(2H, dd, J=13.5, 6.9Hz) | 5.04(dd, J=11.1, 17.4Hz) | 113.0t | 39, 144.2 |
| 2′″ | 6.02(1H, dd, J=6.6, 17.7Hz) | 6.07(dd, J=10.8, 17.4Hz) | 144.2d | 39 |
| 3′″ | — | — | 39.0s | — |
| 4′″ | 1.48(s) | 1.43(d, J=6.6Hz) | 27.2(2C)q | 39, 27.2, 143.6 |
| 5′″ | 1.48(s) | 1.43(d, J=6.6Hz) | | — |

^{a}Measured in DMSO-d₆
*Exchangeable values

In an embodiment of the present invention the compound has been isolated from a mangrove-associated fungus Penicillium chrysogenum. This fungus was identified from Agharkar Research Institute, Pune, India. The said fungus is known and available in public domain. The specific strain isolated and used in the present invention bears reference number FMB 005. It has also been deposited at Microbial Type Culture Collection & Gene Bank, Institute of Microbial Technology, Sector 39-A, Chandigarh—160 036 at Accession number MTCC 5108.

The organism was obtained from leaves of the mangrove plant Porteresia coarctata (Roxb.). The leaves were collected from Chorao Island along the Mandovi estuary of Goa, India, in sterile polythene bags and transported to the laboratory. In the laboratory, the leaves were rinsed with sterile seawater to remove adherent particles and detritus material. The leaves were next kept in a sterile, moist chamber for 2 weeks to allow the fungus to grow and sporulate. Fungal hyphae were picked and separately subcultured, repeatedly to obtain pure isolate of the culture.

The spores of Penicillium chrysogenum are produced in chains from flask-shaped cells, which are found at tips of a brush-like aerial structure. The stalk is called the conidiophore and the spore is called conidium. The spores in Penicillium contain a bluish-green pigment, which gives the culture characteristics bluish-green coloration.

In another embodiment, the above culture was initially grown in small Erlenmeyer flask (100 ml) in potato dextrose broth (PDB) prepared in seawater: distilled water (1:1) under shaker conditions. This culture was used as a seed for mass culturing in 5 litre flasks (4 nos.) containing 1 lit fermentation broth in each flask under stationary conditions. In the present experiment, the fungal strain was cultured at 27-30° C. for 15 days. After 15 days, the mycelia were removed by filtration and the broth was separated from the fungal mat.

In yet another embodiment of the present invention, the process for the extraction of the compound from the fermentation broth is described. Chloroform or ethyl acetate may be used for extracting the fermentation broth.

In a preferred embodiment, chloroform was used in the present study to extract the compound of interest. This chloroform filtrate was concentrated under vacuum to obtain crude chloroform extract (30 mg).

In yet another embodiment of the invention, the isolation of the compound from the crude chloroform extract is effected by the use of conventional techniques, such as thin layer chromatography (TLC) and silica gel column chromatography. In a preferred embodiment the crude chloroform extract was chromatographed over silica gel first using petroleum ether:ethyl acetate (with gradual increasing percentage of ethyl acetate) affording fractions yielding impure chrysogenazine.

Further purification of the compound was affected by gel chromatography (Sephadex LH-20) using chloroform:methanol (1:1), to obtain the pure compound as a yellow solid (9 mg).

In another embodiment of this invention, antibacterial activity of chrysogenazine was tested using simple disc diffusion technique (disc containing 5-30 mcg/disc of sample) on agar plated petridishes (Chabbert, 1963; Rinehart et al. 1981). The assay showed the compound to be active against a human pathogen Vibrio cholerae. Degree of sensitivity of chrysogenazine on test organism was determined by measuring the zone of inhibition in millimetres. In addition, standard discs of penicillin (10 units/disc), amphicillin (10 mcg/disc) and streptomycin (10 mcg/disc) were used to compare the sensitivity.

This compound showed an inhibition zone of 4-5 mm, while penicillin showed 0 mm inhibitions zone, amphicillin showed 0 mm inhibition zone, and streptomycin showed 4-5 mm inhibition zone.

The following examples are given by way of illustrations and should not be construed to limit the scope of the present invention.

### EXAMPLE 1

The mangrove plant Porteresia coarctata (Roxb.) was collected from Chorao Island along the Mandovi estuary of Goa. Leaves of the mangrove plant was collected and transported to the laboratory in sterile polythene bags. In the laboratory the leaves were rinsed with sterile seawater to remove adhered particles and detritus material. The leaves were next kept in a moist chamber, using known standard techniques, for 2 weeks, to allow the fungi to grow and sporulate. Repeated subculturing resulted in pure fungal isolate.

### EXAMPLE 2

The growth conditions of the fungal isolate was optimised and grown on potato dextrose agar (PDA) slants (HiMedia Industries Ltd.) and later grown in small Erlenmeyer flasks (100 ml) in potato dextrose broth prepared in seawater: distilled (1:1) under shaker conditions. The culture obtained at the end of 4-5 days was used to seed 5 lit. Erlenmeyer flasks containing 1 lit of the same medium prepared similarly in replicates of four at room temperature (28-30° C.). The flasks were kept stationary for 15 days. At the end of 15 days fungal mycelia were removed by filtration and fermentation broth was extracted with chloroform.

### EXAMPLE 3

The chloroform extract (30 mg), after removal of the solvent in vacuum, was fractionated through a column of silica gel using petroleum ether:ethyl acetate mixture. Initially, 200 ml of ethyl acetate: petroleum ether in the ratio (1:99%) was used. This was followed by elution with 200ml of a mixture of ethyl acetate: petroleum ether (2:98%). The next percentage of ethyl acetate used was 5% and petroleum ether was 95%. Subsequently, ethyl acetate percentage was increased by 5%. The sub-fractions obtained were spotted on silica gel TLC plates, combined and concentrated after developing and spraying with cerric sulphate.

### EXAMPLE 4

The final purification of the compound was obtained by chromatography using sephadex LH-20 as adsorbent and eluting the compound with chloroform: methanol (1:1). Approximately, 9 mg of chrysogenazine was purified as a yellow solid.

### EXAMPLE 5

In determining the structure of the compound, correlation spectroscopy (COSY), heteronuclear multiple quantum correlation (HMQC), heteronuclear multiple bond correlations (HMBC), distortionless enhancement by polarization transfer (DEPT), ¹H and ¹³C NMR spectral data were obtained using a Brucker Avance 300 Spectrometer. ¹H and ¹³C NMR spectra was recorded at 300 MHz. All the chemical shifts were recorded using TMS as internal standard, at δ 7.24 for proton resonance and δ 77.0 for the carbon spectra. Mass spectral data (ESI-MS) was obtained on a Micro Mass spectrometer; IR spectral data was recorded on FTIR-8201 PC, Shimadzu spectrometer.

Chrysogenazine has the molecular formula of C₁₉H₂₁O₂N₃. Its molecular ion (M⁺) was 323 from (M⁺+Na⁺) and (2M⁺+Na⁺) signals at m/z 346 and 669 respectively.

A close inspection of the ¹H and ¹³C NMR spectra of “1” by DEPT and ¹H-¹³C COSY experiments disclosed signals for 19 carbons: These included one secondary methyl (C-7), two tertiary methyls (C-4′″, C-5′″), one sp³ quarternary carbon (C-3′″), one sp² hybridized methylene (C-1′″), one sp³ hybridized methine (C-6), six sp² methines (C-1′, C-4″, C-5″, C-6″, C-7″ and C-2′″) and seven sp² quarternary carbons including amide carbonyls (C-2, C-3, C-5, C-2″, C-3″, C-3″a and C-7″a). The presence of two secondary amide groups were inferred from signals at 165.5 and 159.6 ppm from its ¹³C NMR spectra (CDCl₃), sharp and strong IR absorptions at 3350 cm⁻¹ and 1676 cm⁻¹, and also from the presence of two D₂O exchangeable protons at δ 6.4 and 7.4 (these signals appeared at δ 8.2 and 8.6 respectively in DMSO). The IR absorption at 1676 cm⁻¹ was also indicative of α-β unsaturated carbonyl functionality. The presence of a third exchangeable proton at δ11.15 in DMSO spectrum and at δ 8.27 in CDCl₃ spectrum along with the pattern of ¹H NMR signals in DMSO (7.47, 7.21, 7.14, 7.06 and 6.96) was suggestive of a conjugated indole nuecleus, as present in dipodazine, (Sorensen et al., 1999) a metabolite from Penicillium dipodomis. The only exception observed was that olefinic methine proton signal at 7.93 of the indole nucleus in dipodazine was absent in chrysogenazine indicating that C-2″ position was also substituted in the latter.

The ¹³C NMR spectrum of dipodazine and chrysogenazine (FIG. 1) are virtually identical with the following changes. The C-2″ carbon at 143 ppm in chrysogenazine is a singlet and has undergone ˜17.0 ppm downfield shift appropriate for tertiary alkyl group substitution (Stothers, 1972). Four new signals (27.2, 39, 113 and 144.2 ppm) have appeared in chrysogenazine spectrum. The intensity of the signal at δ27.2 is suggestive of two similar carbons. These new carbon signals are attributed to an α,α-dimethyl (reversed isopentenyl) substituent which must be attached to the C-2″ of the indole moiety. The cross peaks originating form the vinylic proton ²J_{C-3′″, H-2′″} and ³J_{C-2′″, H-2′″} and ²J_{C-1′″, H-2′″} in HMBC spectrum confirmed the position and the nature of the isopentenyl substitutent (this substituent may also be taken as 1,1 dimethyl-2-propenyl unit).

Considering the formula, the conjugated moiety, isopentenyl substituent and the presence of two secondary amide groups, it was suggestive of tryp-alanine derived cyclic dipeptide. The cross peaks, in the HMBC spectrum, ³J_{C-3″a, H-1′}; ³J_{C-2″, H-1′} and ³J_{C-2, H-1′} connected C-1′ to the indole and diketopiperazine moieties. HMBC connectivities is also observed with the C-7 secondary methyl and the C-6 methine with the C-5 and C-2 carbonyls of diketopiperazine moiety respectively.

All the above data indicated that chrysogenazine is dipodazine extended by a reversed isopentynyl or 1,1 dimethyl 2-propenyl moiety attached at position 2″ of the pyrazole ring of indole moiety, as shown in 1, and dipodazine is tryp-glycine derived cyclic dipeptide whereas chrysogenazine is tryp-alanine derived cyclic depeptide.

### EXAMPLE 6

This example demonstrates antibacterial activity of chrysogenazine. Antibacterial activity was determined using a Gram negative bacterial strain, Vibrio cholerae, in a agar diffusion assay, essentially as described by Chabbert, (1963) and Rinehart et.al.,(1981). Briefly, nutrient-containing agar plates were seeded with the selected target microorganisms and the disc (loaded with 5-10 mcg/disc of chrysogrnazine) was placed on the surface of the medium. Following an appropriate incubation interval, microbial growth inhibition was visualized and quantified by measuring the clear zone around each disc (Plate 1.). Comparison of this was made with the standard antibiotics (penicillin, amphicillin, and streptomycin).

Advantages of the Present Invention

The process for the extraction and isolation of chrysogenazine is simple and requires minimum purification steps.

P. chrysogenum was associated with the mangrove leaves of the plant Porteresia coarctata, collected from Goa Coast, and is well known to produce several antibiotics, active against Gram-positive bacteria. However, in the present invention, chrysogenazine is reported to be active against Gram-negative bacteria Vibrio cholerae, causing cholera in humans.

Another advantage is that the yield of the compound may be enhanced by modifying the carbon and nitrogen source in the fermentation broth as well by modifying the laboratory conditions, so as to make it economical/profitable if found suitable for use against pathogens.

References:

Ariyo B., C. Tamerler, C. Bucke, T. Keshavarz, Enhanced penicillin production by oligosaccharides from batch culture of Penicillium chrysogenum in the stirred tank reactors. FEMS Letter Microbiology, 166, (1998), 165-170.
Arzneim Forsch, Vol. 37(4), (1987), 498-502.
Ballio A. and S. Russi, Chromatographic fractionation and chemical characterization of some oligosaccharides synthesized from lactose by P. chrosogenum.
Bergman J. and A. Brynolf. Synthesis of chrysogine, a metabolite of Penicillium chrysogenum and some related 2-substituted-4-(3H)-Quinazolinones. Tetrahedron, 46, (1990), 1295-1310.
Chabbert Y. A. L′antibiogramme coll Technique de base, Ed Touralle, Saint Mande, (1963), 257.
Dinsmore C. J. and D. C. Beshore. Recent advances in the synthesis of diketopiperazine. Tetrahedron 58, (2002), 3297-3312.
Rinehart K. L. Jr., P. D. Shaw, L. S. Schield, J. B. Bloer, G. C. arbour, E. S. Koker, D. Samain, R. E. Schwartz, A. A. Tymiak, D. L. Weller, G. T. Carter, M. H. G. Munro, R. G. Hughes Jr., H. E. Renis, E. B. Swynenberg, D. A. Stringfellow, J. J. Vavra, J. H. Coats, G. E. Zurenko, S. L. Kuentzel, H.Li, G. J.Bakus, R. C. Brusca, L. L. Craft, D. N. Young and J. L. Connor. Marine natural products as sources of antiviral, Antimicrobial and antineoplastic agents. Pure and Applied Chem., 53, (1981), 795-817.
Sorensen D., T. O. Larsen, C. Christophersen, P. H. Nielsen, U. Authoni. Phytochemistry, 51,(1999), 1181-1183.
Stothers J. B. In Carbon-13 NMR Spectroscopy, Academic Press, New York, 1972, P.97
Thykaer J., B. Christensen and J. Nielsen. Metabolic network analysis of an Adipoyl-7-ADCA-Producing strain of P. chrysogenum. Elucidation of Adipate Degradation. Metabolic Engineering 4, (2002) 151-158.
Trifonov L. S., J. H. Bieri, R. Prewo and D. L. Hoesch and D. M. Rast. Isolation and structural elucidation of three metabolites from Verticillium intertextum, Sorbicillin, dihydrosorbicillin and bisvertinoquinol. Tetrahedron 39, (1983) 4243-4256.
Whiteman P. A. and E. P. Abraham. Phenoxymethyl penicillin amidohydrolases from Penicillium chrysogenum FEBS Letters, 394, (1996), 31-33.
U.S. Pat. No. 3,362,956, January 1968, Archer, 260/268
U.S. Pat. No. 5,681,954, October 1997, Yamamoto et. al., 544/114.

1. 3,1′-didehydro-3[2″(3′″,3′″-dimethyl-prop-2-enyl)-3″-indolyl methylene]-6-methylpiperazine-2,5-dione extracted from a mangrove-associated fungus Penicillium chrysogenum having antibacterial activity, represented by a general formula C₁₉H₂₁O₂N₃ and structural formula as shown below:

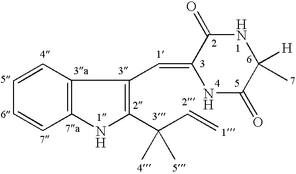

2. A compound as claimed in claim 1, wherein the said compound shows antibacterial activity against the human pathogen Vibrio cholerae.

3. A process of isolation of 3,1′-didehydro-3[2″(3′″,3′″-dimethyl-prop-2-enyl)-3″-indolyl methylene]-6-methylpiperazine-2,5-dione as shown below: 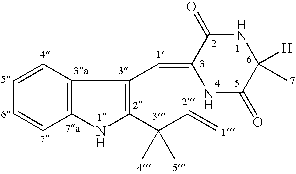
from a fungus Penicillium chrysogenum, said process comprising the steps:
a) growing Penicillium chrysogenum in a fermentation broth comprising potato dextrose agar, sea water and distilled water;
b) extracting the fermentation broth with a solvent to obtain the filtrate;
c) evaporating the filtrate of step (b) to obtain a crude extract;
d) isolating the impure chrysogenazine from the crude extract of step (c) by chromatographic fractionation, and
e) purifying the impure chrysogenazine of step (d) using gel chromatography to obtain the pure chrysogenazine.

4. A process as claimed in claim 3, wherein in step (a), seawater and distilled water is mixed in 1:1 ratio.

5. A process as claimed in claim 3, wherein in step (b), the solvent is selected from a group comprising of chloroform and ethyl acetate.

6. A process as claimed in claim 5, wherein the solvent is chloroform.

7. A process as claimed in claim 3, wherein in step (c), the evaporation is performed under vacuum.

8. A process as claimed in claim 3, wherein in step (d), the chromatographic fractionation is performed by column chromatography and thin layer chromatography.

9. A process as claimed in claim 8, wherein silica gel chromatography is used for fractionation.

10. A process as claimed in claim 9, wherein in silica gel chromatography the eluent used is mixture of petroleum ether and ethyl acetate.

11. A process as claimed in claim 9, wherein in the chromatography the adsorbent used is silica gel with a pore size of 60-120 Å.

12. A process as claimed in claim 3, wherein in step (e), the adsorbent used in gel chromatography is Sephadex LH-20.

13. A process as claimed in claim 3, wherein in step (e), chloroform and methanol mixture is used as an eluent in gel chromatography.

14. A process as claimed in claim 13, wherein the chloroform and methanol are mixed in 1:1 ratio.

15. A process as claimed in claim 13, wherein Penicillium chrysogenum is Penicillium chrysogenum, bearing accession No. MTCC 5108.
